(19) United States
(12) Patent Application Publication
(10) Pub. No.: US 2024/0368558 A1
(43) Pub. Date: Nov. 7, 2024

(54) COMPOSITIONS AND METHODS FOR DECONTAMINATING AND CULTURING A GASTROINTESTINAL TRACT SAMPLE

(71) Applicant: ONCOMEDICS, Limoges (FR)

(72) Inventors: Elodie RIBOT, Limoges (FR); Christophe LAUTRETTE, Limoges (FR); Stéphanie GIRAUD, Limoges (FR)

(73) Assignee: ONCOMEDICS, Limoges (FR)

(21) Appl. No.: 18/293,557

(22) PCT Filed: Jul. 29, 2022
(86) PCT No.: PCT/EP2022/071327

§ 371 Date: Jan. 30, 2024

(30) Foreign Application Priority Data
Jul. 30, 2021 (EP) 21306063.5

(51) Int. Cl. C12N 5/071; C12N 5/09; G01N 33/50

(52) U.S. Cl. CPC C12N 5/0679; C12N 5/0693; G01N 33/5011

(57) ABSTRACT

In the field of gastrointestinal tract samples, in particular of gastrointestinal tract cancer samples, and their decontamination and cultivation. In particular, compositions including a mix of penicillin, streptomycin, gentamicin, ciprofloxacin and vancomycin, capable of efficiently decontaminating a gastrointestinal tract sample while maintaining cells viable. Also, methods using these compositions, for decontaminating a gastrointestinal tract sample, culturing a gastrointestinal tract sample, and evaluating the efficacy of drug candidates against a gastrointestinal tract cancer or tumor.

### FIELD OF INVENTION

The present invention relates to the field of gastrointestinal tract samples, in particular of gastrointestinal tract cancer samples, and their decontamination and cultivation. Disclosed herein in particular are compositions capable of efficiently decontaminating a sample while maintaining cells viable, and methods using the same.

### BACKGROUND OF INVENTION

Chemosensitivity assays, also termed individualized tumor response testing (ITRT), chemotherapy sensitivity and resistance assays (CSRAs), or more generally, functional assays, have been developed for several decades. Assay procedures look at different endpoints, which all share the common feature of being measured on ex vivo models, either whole/minced patient tissue samples, or primary cultures derived from these.

One such functional assay was developed by Oncomedics (Limoges, France) to test the effectiveness of drug candidates and combinations of such candidates for treating breast cancers (EP 1 795 897 B1; Giraud et al., 2011. Anticancer Res. 31(1):139-45; Giraud et al., 2012. Anticancer Res. 32(4):1323-5). It relies on the use of several media for transporting a breast cancer biopsy sample, dissociating the sample, and cultivating it before being contacted with drug candidates.

However, this method suffered drawbacks when it came to adapt it to colorectal cancer biopsy samples. These samples, as well as every gastrointestinal tract sample, are indeed particularly prone to bacterial contamination, because inter alia of their proximity with gut microbiota.

There is thus a need to develop new compositions that allow to efficiently decontaminate gastrointestinal tract samples, and in particular colorectal samples. When used for functional assays, these sample should also remain viable to allow their culture and evaluation of drug candidate efficacy.

The present invention solves these problems, by providing specific compositions capable of efficiently decontaminating colorectal samples while maintaining colorectal tumor cells viable.

### SUMMARY

The present invention relates to a composition comprising penicillin, streptomycin, gentamicin, ciprofloxacin, and vancomycin.

In some embodiments, the composition further comprises a culture medium. In some embodiments, the culture medium is devoid of animal-derived serum or proteins thereof. Alternatively, the culture medium may be supplemented with animal-derived serum or proteins thereof.

In some embodiments, the composition comprises:
penicillin, to a final concentration ranging from about 10 U/mL to about 1000 U/mL;
streptomycin, to a final concentration ranging from about 10 μg/mL to about 1 mg/mL;
gentamicin, to a final concentration ranging from about 10 μg/mL to about 1 mg/mL;
ciprofloxacin, to a final concentration ranging from about 0.5 μg/mL to about 25 μg/mL; and
vancomycin, to a final concentration ranging from about 5 μg/mL to about 125 μg/mL.

In some embodiments, the composition comprises:
penicillin, to a final concentration of about 100 U/mL;
streptomycin, to a final concentration of about 100 μg/mL;
gentamicin, to a final concentration of about 100 μg/mL;
ciprofloxacin, to a final concentration of about 2.5 μg/mL; and
vancomycin, to a final concentration of about 25 μg/mL.

In some embodiments, the composition comprises:
penicillin, to a final concentration of about 1000 U/mL;
streptomycin, to a final concentration of about 1 mg/mL;
gentamicin, to a final concentration of about 1 mg/mL;
ciprofloxacin, to a final concentration of about 25 μg/mL; and
vancomycin, to a final concentration of about 125 μg/mL.

In some embodiments, the composition comprises:
penicillin, to a final concentration of about 500 U/mL;
streptomycin, to a final concentration of about 500 μg/mL;
gentamicin, to a final concentration of about 500 μg/mL;
ciprofloxacin, to a final concentration of about 12.5 μg/mL; and
vancomycin, to a final concentration of about 62.5 μg/mL.
   In some embodiments, this composition may further comprise at least one tissue-dissociation enzyme.

In some embodiments, the composition comprises:
penicillin, to a final concentration ranging from about 50 U/mL to about 300 U/mL;
streptomycin, to a final concentration ranging from about 50 μg/mL to about 300 μg/mL;
gentamicin, to a final concentration ranging from about 50 μg/mL to about 300 μg/mL;
ciprofloxacin, to a final concentration ranging from about 1 μg/mL to about 7.5 μg/mL;
vancomycin, to a final concentration ranging from about 10 μg/mL to about 40 μg/mL; and
amphotericin B, to a final concentration ranging from about 1 μg/mL to about 4 μg/mL.

In some embodiments, this composition may comprise:
penicillin, to a final concentration of about 200 U/mL;
streptomycin, to a final concentration of about 200 μg/mL;
gentamicin, to a final concentration of about 200 μg/mL;
ciprofloxacin, to a final concentration of about 5 μg/mL;
vancomycin, to a final concentration of about 25 μg/mL; and
amphotericin B, to a final concentration of about 2.5 μg/mL.

The present invention also relates to a method of decontaminating a gastrointestinal tract sample, comprising the steps of:
(a) optionally, preserving the gastrointestinal tract sample in a preservation solution comprising:
   the composition comprising penicillin, streptomycin, gentamicin, ciprofloxacin, and vancomycin,
   preferably the composition comprising:
      penicillin, to a final concentration ranging from about 10 U/mL to about 1000 U/mL;
      streptomycin, to a final concentration ranging from about 10 μg/mL to about 1 mg/mL;
      gentamicin, to a final concentration ranging from about 10 μg/mL to about 1 mg/mL;
      ciprofloxacin, to a final concentration ranging from about 0.5 μg/mL to about 25 μg/mL; and.
      vancomycin, to a final concentration ranging from about 5 μg/mL to about 125 μg/mL,
preferably for a period of time of at most about 55 hours and at a temperature ranging from about 2° C. to about 8° C.;
(b) contacting the gastrointestinal tract sample with a first decontamination solution comprising metronidazole, preferably the first decontamination solution comprises from about 1 μg/mL to about 200 μg/mL of metronidazole, preferably contacting is carried out for a period of time ranging from about 10 minutes to about 2 hours at a temperature ranging from about 20° C. to about 42° C. under a controlled atmosphere with a carbon dioxide (CO₂) concentration ranging from about 0.1% to about 20%; and
(c) contacting the gastrointestinal tract sample with a second decontamination solution comprising:
   the composition comprising penicillin, streptomycin, gentamicin, ciprofloxacin, and vancomycin,
   preferably the composition comprising:
      penicillin, to a final concentration ranging from about 10 U/mL to about 1000 U/mL;
      streptomycin, to a final concentration ranging from about 10 μg/mL to about 1 mg/mL;
      gentamicin, to a final concentration ranging from about 10 μg/mL to about 1 mg/mL;
      ciprofloxacin, to a final concentration ranging from about 0.5 μg/mL to about 25 μg/mL; and
      vancomycin, to a final concentration ranging from about 5 μg/mL to about 125 μg/mL,
preferably for a period of time ranging from about 10 minutes to about 2 hours at a temperature ranging from about 20° C. to about 42° C. under a controlled atmosphere with a carbon dioxide (CO₂) concentration ranging from about 0.1% to about 20%, preferably step (c) is repeated twice.

In some embodiments, the method comprises the steps of:
(a) optionally, preserving the gastrointestinal tract sample in a preservation solution comprising the composition comprising:
   penicillin, to a final concentration of about 100 U/mL;
   streptomycin, to a final concentration of about 100 μg/mL;
   gentamicin, to a final concentration of about 100 μg/mL;
   ciprofloxacin, to a final concentration of about 2.5 μg/mL; and
   vancomycin, to a final concentration of about 25 μg/mL,
for a period of time ranging from a few minutes to about 55 hours and at a temperature ranging from about 2° C. to about 8° C.;
(b) contacting the gastrointestinal tract sample with a first decontamination solution comprising metronidazole to a final concentration of about 20 μg/mL, for a period of time ranging from about 10 minutes to about 2 hours at a temperature ranging from about 35° C. to about 42° C. under a controlled atmosphere with a carbon dioxide (CO₂) concentration ranging from about 1% to about 10%; and
(c) contacting the gastrointestinal tract sample with a second decontamination solution comprising the composition comprising:
   penicillin, to a final concentration of about 1000 U/mL;
   streptomycin, to a final concentration of about 1 mg/mL;
   gentamicin, to a final concentration of about 1 mg/mL;
   ciprofloxacin, to a final concentration of about 25 μg/mL; and
   vancomycin, to a final concentration of about 125 μg/mL,
for a period of time ranging from about 10 minutes to about 2 hours at a temperature ranging from about 35° C. to about 42° C. under a controlled atmosphere with a carbon dioxide (CO₂) concentration ranging from about 1% to about 10%, wherein step (c) is repeated twice.

The present invention also relates to a method of culturing a gastrointestinal tract sample, comprising the steps of:
(a) performing the method of decontaminating a gastrointestinal tract sample of the invention;
(b) optionally, mechanically and/or enzymatically dissociating the gastrointestinal tract sample; and
(c) culturing the gastrointestinal tract sample in a culture medium comprising the composition comprising:
   penicillin, to a final concentration ranging from about 50 U/mL to about 300 U/mL;
   streptomycin, to a final concentration ranging from about 50 μg/mL to about 300 μg/mL;
   gentamicin, to a final concentration ranging from about 50 μg/mL to about 300 μg/mL;
   ciprofloxacin, to a final concentration ranging from about 1 μg/mL to about 7.5 μg/mL;
   vancomycin, to a final concentration ranging from about 10 μg/mL to about 40 μg/mL; and
   amphotericin B, to a final concentration ranging from about 1 μg/mL to about 4 μg/mL,
   preferably for several days at a temperature ranging from about 20° C. to about 42° C. under a controlled atmosphere with a carbon dioxide (CO₂) concentration ranging from about 0.1% to about 20%.

In some embodiments, the method comprises the steps of:
(a) performing the method of decontaminating a gastrointestinal tract sample of the invention;
(b) mechanically and enzymatically dissociating the gastrointestinal tract sample, wherein enzymatically dissociating the gastrointestinal tract sample is carried out in the composition comprising:
   penicillin, to a final concentration of about 500 U/mL;
   streptomycin, to a final concentration of about 500 μg/mL;
   gentamicin, to a final concentration of about 500 μg/mL;
   ciprofloxacin, to a final concentration of about 12.5 μg/mL;
   vancomycin, to a final concentration of about 62.5 μg/mL; and
   at least one tissue-dissociation enzyme,
preferably wherein the at least one tissue-dissociation enzyme comprises type II collagenase and/or trypsin; and
(c) culturing the gastrointestinal tract sample in a culture medium comprising the composition comprising:
   penicillin, to a final concentration of about 200 U/mL;
   streptomycin, to a final concentration of about 200 μg/mL;
   gentamicin, to a final concentration of about 200 μg/mL;
   ciprofloxacin, to a final concentration of about 5 μg/mL;
   vancomycin, to a final concentration of about 25 μg/mL; and
   amphotericin B, to a final concentration of about 2.5 μg/mL,
for several days at a temperature ranging from about 35° C. to about 42° C. under a controlled atmosphere with a carbon dioxide (CO₂) concentration ranging from about 1% to about 10%.

The present invention also relates to a method of evaluating the efficacy of one or several drug candidates against a gastrointestinal tract cancer or tumor, comprising the steps of:
(a) performing the method of culturing a gastrointestinal tract sample of the invention, wherein the gastrointestinal tract sample is a gastrointestinal tract cancer or tumor sample;
(b) contacting the gastrointestinal tract sample with the one or several drug candidates;
(c) counting the number of dead cells and optionally, determining a percentage or ratio of dead cells in the gastrointestinal tract sample; and
(d) concluding that the one or several drug candidates is efficient against the gastrointestinal tract cancer or tumor based on the number, percentage and/or ratio of dead cells in the gastrointestinal tract sample.

In some embodiments, step (c) first comprises contacting the gastrointestinal tract sample with a dye, preferably a fluorescent label, specific for living cells, and/or a dye, preferably a fluorescent label, specific for dead cells, and then counting the number of living and/or dead cells based on the color transmitted or emitted by the dye, preferably by the fluorescent label.

In some embodiments applicable to all methods disclosed herein, the gastrointestinal tract sample is a colorectal cancer or tumor sample.

The present invention also relates to a kit-of-parts comprising:
optionally, a preservation medium comprising a composition comprising:
   penicillin, to a final concentration of about 100 U/mL;
   streptomycin, to a final concentration of about 100 μg/mL;
   gentamicin, to a final concentration of about 100 μg/mL;
   ciprofloxacin, to a final concentration of about 2.5 μg/mL; and
   vancomycin, to a final concentration of about 25 μg/mL,
metronidazole;
a decontamination medium comprising a composition comprising:
   penicillin, to a final concentration of about 1000 U/mL;
   streptomycin, to a final concentration of about 1 mg/mL;
   gentamicin, to a final concentration of about 1 mg/ml;
   ciprofloxacin, to a final concentration of about 25 μg/mL; and
   vancomycin, to a final concentration of about 125 μg/mL,
optionally, a dissociation medium comprising a composition comprising:
   penicillin, to a final concentration of about 500 U/mL;
   streptomycin, to a final concentration of about 500 μg/mL;
   gentamicin, to a final concentration of about 500 μg/mL;
   ciprofloxacin, to a final concentration of about 12.5 μg/mL; and
   vancomycin, to a final concentration of about 62.5 μg/mL,
optionally, at least one tissue-dissociation enzyme;
optionally, a culture medium comprising a composition comprising:
   penicillin, to a final concentration of about 200 U/mL;
   streptomycin, to a final concentration of about 200 μg/mL;
   gentamicin, to a final concentration of about 200 μg/mL;
   ciprofloxacin, to a final concentration of about 5 μg/mL;
   vancomycin, to a final concentration of about 25 μg/mL; and
   amphotericin B, to a final concentration of about 2.5 μg/mL, and
optionally, a dye, preferably a fluorescent label, specific for living cells and/or a dye, preferably a fluorescent label, specific for dead cells.

### Definitions

In the present invention, the following terms have the following meanings.

“A” or “an”, refer to one or to more than one (i.e., at least one).

“About”, preceding a figure, means±10% of the value of said figure.

“Basal medium” or “basal culture medium” or “serum-free culture medium” refer to an unsupplemented medium (i.e., lacking protein supplementation, in particular lacking animal-derived serum or proteins thereof, such as FBS or BSA) comprising minimally-required nutrients to maintain the survival of mammalian cells in vitro in a manner that mimics the ideal physiological environment in vivo. Ideal basal media formulations contain chemically-defined components that fulfill optimal conditions and concentrations for pH, proteinogenic amino acids, sugar(s) (such as glucose), water-soluble vitamins, salts and ions, but typically lack growth factors, hormones and lipids. Examples of basal media include, but are not limited to, Harry Eagle's Minimal Essential Media (MEM), α-MEM, Basal Medium Eagle (BME), Glasgow's Minimal Essential Medium (G-MEM), Dulbecco's Modified Eagle Medium (DMEM), Ham's F-12, DMEM/F-12, Roswell Park Memorial Institute medium (RMPI 1640), Iscove's Modified Dulbecco's Medium (IMDM), Medium 199, Dulbecco's Phosphate Buffered Saline (D-PBS), and Hank's Balanced Salt Solution (HBSS).

“Gastrointestinal tract sample” refers to a sample obtained from the gastrointestinal tract of a mammal, preferably of a human subject. The “gastrointestinal tract” can be divided into upper gastrointestinal tract (which comprises the mouth, pharynx, esophagus, stomach and duodenum), the lower gastrointestinal tract (which comprises the small intestine and the large intestine, the latter being itself subdivided into cecum, ascending colon, right colic flexure, transverse colon, left colic flexure, descending colon, sigmoid colon, rectum and anus). A “gastrointestinal tract sample” is preferably a tissue sample, such as a biopsy sample. In particular, the “gastrointestinal tract sample” may be a tumor sample, such as a tumor biopsy sample, i.e., a sample from a gastrointestinal tract tumor or cancer such as, without limitation, an esophageal cancer, a gastric cancer, a gastrointestinal stromal tumor (GIST), a small intestine cancer, a colorectal cancer, or an anal cancer.

### DETAILED DESCRIPTION

In a first aspect, the present invention relates to compositions comprising a mix of antimicrobial agents.

These compositions are particularly suitable for preserving a gastrointestinal tract sample before processing, for decontaminating a gastrointestinal tract sample in vitro, and/or for cultivating a gastrointestinal tract sample in vitro without microbial contamination and without affecting the viability of the sample's cells.

In one embodiment, the composition comprises penicillin, streptomycin, gentamicin, ciprofloxacin, and vancomycin.

In one embodiment, the composition comprises a culture medium, such as a basal medium, as well as penicillin, streptomycin, gentamicin, ciprofloxacin, and vancomycin.

In one embodiment, the culture medium is a basal medium selected from the group consisting of MEM, α-MEM, BME, G-MEM, DMEM, F-12, DMEM/F-12, RMPI 1640, IMDM, Medium 199, D-PBS, and HBSS. In one embodiment, the basal medium is DMEM/F-12.

In one embodiment, the composition comprises:
optionally, a culture medium, such as a basal culture medium;
penicillin, to a final concentration ranging from about 10 U/mL to about 1000 U/mL, preferably from about 100 U/mL to about 1000 U/mL;
streptomycin, to a final concentration ranging from about 10 μg/mL to about 1 mg/mL, preferably from about 100 μg/mL to about 1000 μg/mL;
gentamicin, to a final concentration ranging from about 10 μg/mL to about 1 mg/mL, preferably from about 100 μg/mL to about 1 mg/mL;
ciprofloxacin, to a final concentration ranging from about 0.5 μg/mL to about 25 μg/mL, preferably from about 2.5 μg/mL to about 25 μg/mL; and
vancomycin, to a final concentration ranging from about 5 μg/mL to about 125 μg/mL, preferably from about 25 μg/mL to about 125 μg/mL.

In one embodiment, the composition comprises:
optionally, a culture medium, such as a basal culture medium;
penicillin, to a final concentration of about 100 U/mL;
streptomycin, to a final concentration of about 100 μg/mL;
gentamicin, to a final concentration of about 100 μg/mL;
ciprofloxacin, to a final concentration of about 2.5 μg/mL; and
vancomycin, to a final concentration of about 25 μg/mL.

In one embodiment, the composition comprises:
optionally, a culture medium, such as a basal culture medium;
penicillin, to a final concentration of about 1000 U/mL;
streptomycin, to a final concentration of about 1 mg/mL;
gentamicin, to a final concentration of about 1 mg/mL;
ciprofloxacin, to a final concentration of about 25 μg/mL; and
vancomycin, to a final concentration of about 125 μg/mL.

In one embodiment, the composition comprises:
optionally, a culture medium, such as a basal culture medium;
penicillin, to a final concentration of about 500 U/mL;
streptomycin, to a final concentration of about 500 μg/mL;
gentamicin, to a final concentration of about 500 μg/mL;
ciprofloxacin, to a final concentration of about 12.5 μg/mL; and
vancomycin, to a final concentration of about 62.5 μg/mL.

In one embodiment, the composition comprises:
optionally, a culture medium, such as a basal culture medium;
penicillin, to a final concentration of about 200 U/mL;
streptomycin, to a final concentration of about 200 μg/mL;
gentamicin, to a final concentration of about 200 μg/mL;
ciprofloxacin, to a final concentration of about 5 μg/mL; and vancomycin, to a final concentration of about 25 μg/mL.

In one embodiment, the composition further comprises amphotericin B. In one embodiment, the composition further comprises amphotericin B to a final concentration ranging from about 0.5 μg/mL to about 25 μg/mL, preferably from about 0.5 μg/mL to about 10 μg/mL. In one embodiment, the composition further comprises amphotericin B to a final concentration of about 2.5 μg/mL.

In one embodiment, the composition comprises:
optionally, a culture medium, such as a basal culture medium;
penicillin, to a final concentration ranging from about 10 U/mL to about 400 U/mL, preferably from about 50 U/mL to about 300 U/mL, more preferably from about 100 U/mL to about 300 U/mL;
streptomycin, to a final concentration ranging from about 10 μg/mL to about 400 μg/mL, preferably from about 50 μg/mL to about 300 μg/mL, more preferably from about 100 μg/mL to about 300 μg/mL;

gentamicin, to a final concentration ranging from about 10 μg/mL to about 400 μg/mL, preferably from about 50 μg/mL to about 300 μg/mL, more preferably from about 100 μg/mL to about 300 μg/mL;
ciprofloxacin, to a final concentration ranging from about 0.5 μg/mL to about 10 μg/mL, preferably from about 1 μg/mL to about 7.5 μg/mL, more preferably from about 2.5 μg/mL to about 7.5 μg/mL;
vancomycin, to a final concentration ranging from about 5 μg/mL to about 50 μg/mL, preferably from about 10 μg/mL to about 40 μg/mL; and
amphotericin B, to a final concentration ranging from about 0.5 μg/mL to about 5 μg/mL, preferably from about 1 μg/mL to about 4 μg/mL.

In one embodiment, the composition comprises:
optionally, a culture medium, such as a basal culture medium;
penicillin, to a final concentration of about 200 U/mL;
streptomycin, to a final concentration of about 200 μg/mL;
gentamicin, to a final concentration of about 200 μg/mL;
ciprofloxacin, to a final concentration of about 5 μg/mL;
vancomycin, to a final concentration of about 25 μg/mL; and
amphotericin B, to a final concentration of about 2.5 μg/mL.

In one embodiment, the composition may further comprise a protein supplementation. Examples of suitable protein supplementations include, but are not limited to, bovine serum albumin (BSA), fetal bovine serum (FBS), and fetal calf serum (FCS).

In one embodiment, the composition may further comprise a protein supplementation, in particular BSA, to a final concentration ranging from about 0.1 mg/mL to about 10 mg/mL, preferably from about 0.5 mg/mL to about 5 mg/mL, more preferably from about 1 mg/mL to about 2 mg/mL. In one embodiment, the composition may further comprise a protein supplementation, in particular BSA, to a final concentration of about 1 mg/mL or about 2 mg/mL.

In a second aspect, the invention relates to a method of decontaminating a gastrointestinal tract sample.

In one embodiment, the gastrointestinal tract sample is a tissue sample, in particular a biopsy sample, more particularly a tumor biopsy sample, such as tumor biopsy sample from an esophageal cancer, a gastric cancer, a gastrointestinal stromal tumor (GIST), a small intestine cancer, a colorectal cancer, or an anal cancer.

In a preferred embodiment, the gastrointestinal tract sample is a colorectal cancer biopsy sample.

In one embodiment, the method is an in vitro method. In one embodiment, the method does not comprise a step of collecting the gastrointestinal tract sample from a subject.

In one embodiment, the method may comprise a preliminary step of preserving the gastrointestinal tract sample in a preservation solution prior to performing the decontamination step described below. This preliminary step is particularly useful when the gastrointestinal tract sample is not or cannot be processed immediately (such as, in the first few minutes to hours) after collection.

In one embodiment, the preservation solution comprises a culture medium, such as a basal medium, as well as penicillin, streptomycin, gentamicin, ciprofloxacin, and vancomycin.

In one embodiment, the culture medium is a basal medium selected from the group consisting of MEM, α-MEM, BME, G-MEM, DMEM, F-12, DMEM/F-12, RMPI 1640, IMDM, Medium 199, D-PBS, and HBSS. In one embodiment, the basal medium is DMEM/F-12.

In one embodiment, the preservation solution comprises:
optionally, a culture medium, such as a basal culture medium;
penicillin, to a final concentration ranging from about 10 U/mL to about 1000 U/mL, preferably from about 100 U/mL to about 1000 U/mL;
streptomycin, to a final concentration ranging from about 10 μg/mL to about 1 mg/mL, preferably from about 100 μg/mL to about 1000 μg/mL;
gentamicin, to a final concentration ranging from about 10 μg/mL to about 1 mg/mL, preferably from about 100 μg/mL to about 1 mg/mL;
ciprofloxacin, to a final concentration ranging from about 0.5 μg/mL to about 25 μg/mL, preferably from about 2.5 μg/mL to about 25 μg/mL; and
vancomycin, to a final concentration ranging from about 5 μg/mL to about 125 μg/mL, preferably from about 25 μg/mL to about 125 μg/mL.

In one embodiment, the preservation solution comprises:
optionally, a culture medium, such as a basal culture medium;
penicillin, to a final concentration of about 100 U/mL;
streptomycin, to a final concentration of about 100 μg/mL;
gentamicin, to a final concentration of about 100 μg/mL;
ciprofloxacin, to a final concentration of about 2.5 μg/mL; and
vancomycin, to a final concentration of about 25 μg/mL.

In one embodiment, the gastrointestinal tract sample can be contacted with the preservation solution and stored for a period of time ranging from a few minutes to about 55 hours.

In one embodiment, the gastrointestinal tract sample can be contacted with the preservation solution and stored at a temperature ranging from about 2° C. to about 8° C., preferably at about 4° C. or at refrigerator temperature as defined by the European Pharmacopoeia in Chapter I.2 “Other provisions applying to general chapters and monographs”.

In one embodiment, the method comprises a step of contacting the gastrointestinal tract sample with a first decontamination solution comprising a culture medium, such as a basal medium, and metronidazole.

In one embodiment, the culture medium is a basal medium selected from the group consisting of MEM, α-MEM, BME, G-MEM, DMEM, F-12, DMEM/F-12, RMPI 1640, IMDM, Medium 199, D-PBS, and HBSS. In one embodiment, the basal medium is DMEM/F-12.

In one embodiment, the first decontamination solution comprises metronidazole to a final concentration ranging from about 1 μg/mL to about 200 μg/mL, preferably from about 5 μg/mL to about 100 μg/mL. In one embodiment, the first decontamination solution comprises metronidazole to a final concentration of about 20 μg/mL.

In one embodiment, the gastrointestinal tract sample is contacted with the first decontamination solution for a period of time ranging from about 10 minutes to about 2 hours, preferably from about 10 minutes to about 1 hour, more preferably for about 30 minutes.

In one embodiment, the gastrointestinal tract sample is contacted with the first decontamination solution at a temperature ranging from about 20° C. to about 42° C., preferably from about 35° C. to about 42° C., more preferably at about 37° C.

In one embodiment, the gastrointestinal tract sample is contacted with the first decontamination solution under a controlled atmosphere with a carbon dioxide (CO₂) concentration ranging from about 0.1% to about 20%, preferably from about 1% to about 10%, more preferably under a controlled atmosphere with about 5% CO₂.

In one embodiment, the gastrointestinal tract sample is contacted once, twice, three times or more with the first decontamination solution, preferably the gastrointestinal tract sample is contacted once with the first decontamination solution. In one embodiment, the first decontamination solution is replenished between each of the two, three or more contacts with the gastrointestinal tract sample. Preferably, a fresh first decontamination solution is used for the second, third or more contact with the gastrointestinal tract sample.

In a preferred embodiment, the gastrointestinal tract sample is contacted once with the first decontamination solution comprising a culture medium, such as a basal medium, and metronidazole to a final concentration of about 20 μg/mL, for a period of time of about 30 minutes, at a temperature of about 37° C., under a controlled atmosphere with about 5% CO₂.

In one embodiment, the method comprises a step of contacting the gastrointestinal tract sample with a second decontamination solution comprising a culture medium, such as a basal medium, as well as penicillin, streptomycin, gentamicin, ciprofloxacin, and vancomycin.

In one embodiment, the culture medium is a basal medium selected from the group consisting of MEM, α-MEM, BME, G-MEM, DMEM, F-12, DMEM/F-12, RMPI 1640, IMDM, Medium 199, D-PBS, and HBSS. In one embodiment, the basal medium is DMEM/F-12.

In one embodiment, the second decontamination solution comprises:
optionally, a culture medium, such as a basal culture medium;
penicillin, to a final concentration ranging from about 10 U/mL to about 1000 U/mL, preferably from about 100 U/mL to about 1000 U/mL;
streptomycin, to a final concentration ranging from about 10 μg/mL to about 1 mg/mL, preferably from about 100 μg/mL to about 1000 μg/mL;
gentamicin, to a final concentration ranging from about 10 μg/mL to about 1 mg/mL, preferably from about 100 μg/mL to about 1 mg/mL;
ciprofloxacin, to a final concentration ranging from about 0.5 μg/mL to about 25 μg/mL, preferably from about 2.5 μg/mL to about 25 μg/mL; and
vancomycin, to a final concentration ranging from about 5 μg/mL to about 125 μg/mL, preferably from about 25 μg/mL to about 125 μg/mL.

In one embodiment, the second decontamination solution comprises:
optionally, a culture medium, such as a basal culture medium;
penicillin, to a final concentration of about 1000 U/mL;
streptomycin, to a final concentration of about 1 mg/mL;
gentamicin, to a final concentration of about 1 mg/mL;
ciprofloxacin, to a final concentration of about 25 μg/mL; and
vancomycin, to a final concentration of about 125 μg/mL.

In one embodiment, the gastrointestinal tract sample is contacted with the second decontamination solution for a period of time ranging from about 10 minutes to about 2 hours, preferably from about 10 minutes to about 1 hour, more preferably for about 30 minutes.

In one embodiment, the gastrointestinal tract sample is contacted with the second decontamination solution at a temperature ranging from about 20° C. to about 42° C., preferably from about 35° C. to about 42° C., more preferably at about 37° C.

In one embodiment, the gastrointestinal tract sample is contacted with the second decontamination solution under a controlled atmosphere with a carbon dioxide (CO₂) concentration ranging from about 0.1% to about 20%, preferably from about 1% to about 10%, more preferably under a controlled atmosphere with about 5% CO₂.

In one embodiment, the gastrointestinal tract sample is contacted once, twice, three times or more with the second decontamination solution, preferably the gastrointestinal tract sample is contacted twice with the second decontamination solution. In one embodiment, the second decontamination solution is replenished between each of the two, three or more contacts with the gastrointestinal tract sample. In one embodiment, the second decontamination solution is removed and fleshly replaced with a new second decontamination solution for the second, third or more contact with the gastrointestinal tract sample. Alternatively, the gastrointestinal tract sample is removed from the second decontamination solution and placed in a new second decontamination solution for the second, third or more contact.

In a preferred embodiment, the gastrointestinal tract sample is contacted twice with the second decontamination solution comprising a culture medium, such as a basal medium, as well as penicillin to a final concentration of about 1000 U/mL, streptomycin to a final concentration of about 1 mg/mL, gentamicin to a final concentration of about 1 mg/mL, ciprofloxacin to a final concentration of about 25 μg/mL, and vancomycin to a final concentration of about 125 μg/mL, for a period of time of about 30 minutes/contact (i.e., 1 hour total), at a temperature of about 37° C., under a controlled atmosphere with about 5% CO₂.

In a third aspect, the invention relates to a method of culturing a gastrointestinal tract sample.

In one embodiment, the gastrointestinal tract sample is a tissue sample, in particular a biopsy sample, more particularly a tumor biopsy sample, such as tumor biopsy sample from an esophageal cancer, a gastric cancer, a gastrointestinal stromal tumor (GIST), a small intestine cancer, a colorectal cancer, or an anal cancer.

In a preferred embodiment, the gastrointestinal tract sample is a colorectal cancer biopsy sample.

In one embodiment, the method may comprise a preliminary step of preserving the gastrointestinal tract sample in a preservation solution prior to performing the culturing step described below. This preliminary step is particularly useful when the gastrointestinal tract sample is not or cannot be processed immediately (such as, in the first few minutes to hours) after collection. This step of preserving the gastrointestinal tract sample in a preservation solution was described above, as part of the method of decontaminating a gastrointestinal tract sample, which applies mutatis mutandis to the present method.

In one embodiment, the method may comprise a step of contacting the gastrointestinal tract sample with a first decontamination solution comprising a culture medium, such as a basal medium, and metronidazole prior to performing the culturing step described below. This step of contacting the gastrointestinal tract sample with a first decontamination solution was described above, as part of the method of decontaminating a gastrointestinal tract sample, which applies mutatis mutandis to the present method.

In one embodiment, the method may comprise a step of contacting the gastrointestinal tract sample with a second decontamination solution comprising a culture medium, such as a basal medium, as well as penicillin, streptomycin, gentamicin, ciprofloxacin, and vancomycin prior to performing the culturing step described below.

This step of contacting the gastrointestinal tract sample with a second decontamination solution was described above, as part of the method of decontaminating a gastrointestinal tract sample, which applies mutatis mutandis to the present method.

In one embodiment, the method may comprise a step of dissociating the gastrointestinal tract sample prior to performing the culturing step described below.

In one embodiment, the step of dissociating the gastrointestinal tract sample may comprise a substep of mechanically dissociating the gastrointestinal tract sample. By “mechanically dissociating” the gastrointestinal tract sample, it is meant cutting the gastrointestinal tract sample into fragments, by means, e.g., of a knife, scalpel, tissue chopper, or any other suitable cutting device. Fragments should preferably have an average size ranging from about 5 mm³ to about 10 mm³.

In one embodiment, the step of dissociating the gastrointestinal tract sample may comprise a substep of enzymatically dissociating the gastrointestinal tract sample in a dissociation solution. By “enzymatically dissociating” the gastrointestinal tract sample, it is meant contacting the gastrointestinal tract sample with enzymes capable of releasing cells from a tissue. These enzymes are referred to in the art as “tissue-dissociation enzymes” Enzymes suitable for enzymatically dissociating a gastrointestinal tract sample are typically proteases, and include, without limitation, type I collagenase, type II collagenase, trypsin, thermolysin, dispase, hyaluronidase, papain, clostripain, and combinations thereof.

In one embodiment, the dissociation solution comprises:
optionally, a culture medium, such as a basal culture medium;
penicillin, to a final concentration ranging from about 10 U/mL to about 1000 U/mL, preferably from about 100 U/mL to about 1000 U/mL;
streptomycin, to a final concentration ranging from about 10 μg/mL to about 1 mg/mL, preferably from about 100 μg/mL to about 1000 μg/mL;
gentamicin, to a final concentration ranging from about 10 μg/mL to about 1 mg/mL, preferably from about 100 μg/mL to about 1 mg/mL;
ciprofloxacin, to a final concentration ranging from about 0.5 μg/mL to about 25 μg/mL, preferably from about 2.5 μg/mL to about 25 μg/mL;
vancomycin, to a final concentration ranging from about 5 μg/mL to about 125 μg/mL, preferably from about 25 μg/mL to about 125 μg/mL; and
at least one enzyme capable of releasing cells from a tissue (i.e., a tissue-dissociation enzyme).

In one embodiment, the dissociation solution comprises:
optionally, a culture medium, such as a basal culture medium;
penicillin, to a final concentration of about 500 U/mL;
streptomycin, to a final concentration of about 500 μg/mL;
gentamicin, to a final concentration of about 500 μg/mL;
ciprofloxacin, to a final concentration of about 12.5 μg/mL;
vancomycin, to a final concentration of about 62.5 μg/mL; and
at least one enzyme capable of releasing cells from a tissue (i.e., a tissue-dissociation enzyme).

In one embodiment, the at least one enzyme capable of releasing cells from a tissue (i.e., the tissue-dissociation enzyme) is selected from the group comprising or consisting of type I collagenase, type II collagenase, trypsin, thermolysin, dispase, hyaluronidase, papain, and clostripain. In one embodiment, the at least one enzyme capable of releasing cells from a tissue (i.e., the tissue-dissociation enzyme) is selected from type II collagenase and trypsin. In one embodiment, the dissociation solution comprises type II collagenase and trypsin.

In one embodiment, the substep of enzymatically dissociating the gastrointestinal tract sample for a period of time ranging from about 30 minutes to about 4 hours, preferably from about 90 minutes to about 150 minutes.

In a preferred embodiment, the step of dissociating the gastrointestinal tract sample comprises a first substep of mechanically dissociating the gastrointestinal tract sample, followed by a second substep of enzymatically dissociating the gastrointestinal tract sample.

After this step of dissociating the gastrointestinal tract sample, the sample can be referred to a gastrointestinal tract cell sample.

In one embodiment, the method comprises a step of culturing the gastrointestinal tract sample in a culture medium, such as a basal medium, comprising penicillin, streptomycin, gentamicin, ciprofloxacin, vancomycin and amphotericin B.

In one embodiment, the culture medium is a basal medium selected from the group consisting of MEM, α-MEM, BME, G-MEM, DMEM, F-12, DMEM/F-12, RMPI 1640, IMDM, Medium 199, D-PBS, and HBSS. In one embodiment, the basal medium is DMEM/F-12.

In one embodiment, the culture medium comprises:
a culture medium, such as a basal culture medium;
penicillin, to a final concentration ranging from about 10 U/mL to about 400 U/mL, preferably from about 50 U/mL to about 300 U/mL, more preferably from about 100 U/mL to about 300 U/mL;
streptomycin, to a final concentration ranging from about 10 μg/mL to about 400 μg/mL, preferably from about 50 μg/mL to about 300 μg/mL, more preferably from about 100 μg/mL to about 300 μg/mL;
gentamicin, to a final concentration ranging from about 10 μg/mL to about 400 μg/mL, preferably from about 50 μg/mL to about 300 μg/mL, more preferably from about 100 μg/mL to about 300 μg/mL;
ciprofloxacin, to a final concentration ranging from about 0.5 μg/mL to about 10 μg/mL, preferably from about 1 μg/mL to about 7.5 μg/mL, more preferably from about 2.5 μg/mL to about 7.5 μg/mL;
vancomycin, to a final concentration ranging from about 5 μg/mL to about 50 μg/mL, preferably from about 10 μg/mL to about 40 μg/mL; and
amphotericin B, to a final concentration ranging from about 0.5 μg/mL to about 5 μg/mL, preferably from about 1 μg/mL to about 4 μg/mL.

In one embodiment, the culture medium comprises:
a culture medium, such as a basal culture medium;
penicillin, to a final concentration of about 200 U/mL;
streptomycin, to a final concentration of about 200 μg/mL;
gentamicin, to a final concentration of about 200 μg/mL;
ciprofloxacin, to a final concentration of about 5 μg/mL;
vancomycin, to a final concentration of about 25 μg/mL; and
amphotericin B, to a final concentration of about 2.5 μg/mL.

In one embodiment, the gastrointestinal tract sample is cultured for several days, such as 1, 2, 3, 4, 5, 6, 7, 8, 9, 10, 11, 12, 13, 14 days or more. In one embodiment, the gastrointestinal tract sample is cultured for a period of time ranging from 5 to 8 days.

In one embodiment, the gastrointestinal tract sample is cultured at a temperature ranging from about 20° C. to about 42° C., preferably from about 35° C. to about 42° C., more preferably at about 37° C.

In one embodiment, the gastrointestinal tract sample is cultured under a controlled atmosphere with a carbon dioxide (CO₂) concentration ranging from about 0.1% to about 20%, preferably from about 1% to about 10%, more preferably under a controlled atmosphere with about 5% CO₂.

In one embodiment, the culture medium is replenished, or alternatively is removed and fleshly replaced with a new culture medium every 2, 3, 4, 5, 6, 7 days or more, preferably every 5 days.

The skilled artisan is familiar with cell culture protocols and will readily appreciate the various parameters such as duration, temperature, CO₂ concentration, humidity, etc.

In a fourth aspect, the invention relates to a method of evaluating drug candidate efficacy.

This method is particularly appropriate when the gastrointestinal tract sample is a gastrointestinal tract tumor biopsy sample, such as tumor biopsy sample from an esophageal cancer, a gastric cancer, a gastrointestinal stromal tumor (GIST), a small intestine cancer, a colorectal cancer, or an anal cancer.

In a preferred embodiment, the gastrointestinal tract sample is a colorectal cancer biopsy sample.

In one embodiment, the method comprises, as a preliminary step, performing the method of culturing a gastrointestinal tract sample described above.

In one embodiment, the method comprises a step of evaluating the therapeutic efficacy of drug candidates against cancer cells from the gastrointestinal tract tumor biopsy sample.

In one embodiment, the step of evaluating the therapeutic efficacy of drug candidates comprises contacting the gastrointestinal tract sample-or the cancer cells from the gastrointestinal tract sample-with drug candidates. A gastrointestinal tract sample can be contacted with one drug candidate or with combinations or two or more drug candidates. Several gastrointestinal tract samples can be contacted in parallel, each with a different drug candidate, with different combinations of drug candidates, and/or with the same drug candidate or same combination of drug candidates but at different concentrations.

In one embodiment, the method comprises a step of counting the total number of cells in the gastrointestinal tract sample before contacting it with drug candidates. In one embodiment, the method comprises a step of counting the number of living versus dead cells in the gastrointestinal tract sample before contacting it with drug candidates.

In one embodiment, the drug candidate is selected from the group comprising or consisting of chemotherapeutic agents, targeted therapy agents, cytotoxic agents, cytotoxic agents, pro-apoptotic agents, anti-angiogenic agents, and combinations thereof.

It will be apparent that some drug candidates may fall into more than one category. Although some specific examples of suitable drug candidates will be described in the following, it should be understood that the method described herein is not limited thereto, and can readily apply to any suitable drug candidate for which a therapeutic efficacy against cancer cells from the gastrointestinal tract tumor biopsy sample is to be evaluated, including drugs not yet developed at the time of filing of the present patent application.

In one embodiment, the drug candidate is a chemotherapeutic agent. As used herein, the term “chemotherapeutic agent” refers to any molecule that is effective in inhibiting tumor growth.

Suitable examples of chemotherapeutic agents include those described under subgroup L01 of the Anatomical Therapeutic Chemical Classification System.

Suitable examples of chemotherapeutic agents include, but are not limited to:
(i) alkylating agents, such as, e.g.:
   nitrogen mustards, including chlormethine, cyclophosphamide, ifosfamide, trofosfamide, chlorambucil, melphalan, prednimustine, bendamustine, uramustine, chlornaphazine, cholophosphamide, estrarnustine, mechlorethamine, mechlorethamine oxide hydrochloride, novembichin, phenesterine, uracil mustard and the like;
   nitrosoureas, including carmustine, lomustine, semustine, fotemustine, nimustine, ranimustine, streptozocin, chlorozotocin, and the like;
   alkyl sulfonates, including busulfan, mannosulfan, treosulfan, and the like;
   aziridines, including carboquone, thiotepa, triaziquone, triethylenemelamine, benzodopa, meturedopa, uredopa, and the like; hydrazines, including procarbazine, and the like;
   triazenes, including dacarbazine, temozolomide, and the like; ethylenimines and methylamelamines, including altretamine, triethylenemelamine, trietylenephosphoramide, triethylenethiophosphaorarnide, trimethylolomelamine and the like;
   and others, including mitobronitol, pipobroman, actinomycin, bleomycin, mitomycins (including mitomycin C, and the like), plicamycin, and the like;
(ii) acetogenins, such as, e.g., bullatacin, bullatacinone, and the like;
   (iii) benzodiazepines, such as, e.g., 2-oxoquazepam, 3-hydroxyphenazepam, bromazepam, camazepam, carburazepam, chlordiazepoxide, cinazepam, cinolazepam, clonazepam, cloniprazepam, clorazepate, cyprazepam, delorazepam, demoxepam, desmethylflunitrazepam, devazepide, diazepam, diclazepam, difludiazepam, doxefazepam, elfazepam, ethyl carfluzepate, ethyl dirazepate, ethyl loflazepate, flubromazepam, fletazepam, fludiazepam, flunitrazepam, flurazepam, flutemazepam, flutoprazepam, fosazepam, gidazepam, halazepam, iclazepam, irazepine, kenazepine, ketazolam, lorazepam, lormetazepam, lufuradom, meclonazepam, medazepam, menitrazepam, metaclazepam, motrazepam, N-desalkylflurazepam, nifoxipam, nimetazepam, nitemazepam, nitrazepam, nitrazepate, nordazepam, nortetrazepam, oxazepam, phenazepam, pinazepam, pivoxazepam, prazepam, proflazepam, quazepam, QH-II-66, reclazepam, RO4491533, Ro5-4864, SH-I-048A, sulazepam, temazepam, tetrazepam, tifluadom, tolufazepam, triflunordazepam, tuclazepam, uldazepam, arfendazam, clobazam, CP-1414S, lofendazam, triflubazam, girisopam, GYKI-52466, GYKI-52895, nerisopam, talampanel, tofisopam, adinazolam, alprazolam, bromazolam, clonazolam, estazolam, flualprazolam, flubromazolam, flunitrazolam, nitrazolam, pyrazolam, triazolam, bretazenil, climazolam, EVT-201, FG-8205, flumazenil, GL-II-73, imidazenil, ¹²³I-iomazenil, L-655,708, loprazolam, midazolam, PWZ-029, remimazolam, Ro15-4513, Ro48-6791, Ro48-8684, Ro4938581, sarmazenil, SH-053-R-CH3-2′F, cloxazolam, flutazolam, haloxazolam, mexazolam, oxazolam, bentazepam, clotiazepam, brotizolam, ciclotizolam, deschloroetizolam, etizolam, fluclotizolam, israpafant, JQ1, metizolam, olanzapine, telenzepine, lopirazepam, zapizolam, razobazam, ripazepam, zolazepam, zomebazam, zometapine, premazepam, clazolam, anthramycin, avizafone, rilmazafone, and the like;
(iv) antimetabolites, such as, e.g.:
   antifolates, including aminopterin, methotrexate, pemetrexed, pralatrexate, pteropterin, raltitrexed, denopterin, trimetrexate, pemetrexed, and the like;
   purine analogues, including pentostatin, cladribine, clofarabine, fludarabine, nelarabine, tioguanine, mercaptopurine, and the like;
   pyrimidine analogues, including fluorouracil, capecitabine, doxifluridine, tegafur, tegafur/gimeracil/oteracil, carmofur, floxuridine, cytarabine, gemcitabine, azacytidine, decitabine, and the like; and
   hydroxycarbamide;
(v) androgens, such as, e.g., calusterone, dromostanolone propionate, epitiostanol, mepitiostane, testolactone, and the like;
(vi) anti-adrenals, such as, e.g., aminoglutethimide, mitotane, trilostane, and the like;
(vii) folic acid replenishers, such as, e.g., frolinic acid, and the like;
(viii) maytansinoids, such as, e.g., maytansine, ansamitocins, and the like;
(ix) platinum analogs, such as, e.g., platinum, carboplatin, cisplatin, dicycloplatin, nedaplatin, oxaliplatin, satraplatin, and the like;
(x) antihormonal agents, such as, e.g.:
   anti-estrogens, including tamoxifen, raloxifene, aromatase inhibiting 4(5)-imidazoles, 4-hydroxytamoxifen, trioxifene, keoxifene, LY117018, onapristone, toremifene, and the like;
   anti-androgens, including flutamide, nilutamide, bicalutamide, leuprolide, goserelin, and the like;
(xi) topoisomerase inhibitors, such as, e.g., belotecan, camptothecin, cositecan, etirinotecan pegol, exatecan, gimatecan, irinotecan, lurtotecan, rubitecan, silatecan, topotecan, etoposide, teniposide, aclarubicin, amrubicin, daunorubicin, doxorubicin (including morpholino-doxorubicin, cyanomorpholino-doxorubicin, 2-pyrrolino-doxorubicin, deoxydoxorubicin, and the like), epirubicin, esorubicin, idarubicin, pirarubicin, valrubicin, zorubicin, mitoxantrone, losoxantrone, pixantrone, and the like;
(xii) trichothecenes, such as, e.g., T-2 toxin, verracurin A, roridinA, anguidine and the like;
(xiii) toxoids, such as, e.g., cabazitaxel, docetaxel, larotaxel, ortataxel, paclitaxel, tesetaxel, and the like;
(xiv) others, such as, e.g., bryostatin; callystatin; CC-1065 (including its adozelesin, carzelesin and bizelesin synthetic analogues); cryptophycins (including cryptophycin 1 and cryptophycin 8); dolastatin; duocarmycin (including its synthetic analogues KW-2189 and CBI-TMI); eleutherobin; pancratistatin; sarcodictyin; spongistatin; aclacinomysins; authramycin; azaserine; bleomycin; cactinomycin; carabicin; canninomycin; carzinophilin; chromomycins; dactinomycin; detorubicin; 6-diazo-5-oxo-L-norleucine; marcellomycin; mycophenolic acid; nogalarnycin; olivomycins; peplomycin; potfiromycin; puromycin; quelamycin; rodorubicin; streptomgrin; streptozocin; tubercidin; ubenimex; zinostatin; aceglatone; aldophospharnide glycoside; aminolevulinic acid; amsacrine; bestrabucil; bisantrene; edatraxate; defofamine; demecolcine; diaziquone; elfornithine; elliptinium acetate; epothilone; etoglucid; gallium nitrate; hydroxyurea; lentinan; lonidamine; mitoguazone; mopidamol; nitracrine; phenamet; podophyllinic acid; 2-ethylhydrazide; PSK®; razoxane; rhizoxin; sizofiran; spirogennanium; tenuazonic acid; 2,2′,2″-trichlorotriethylarnine; urethan; vindesine; dacarbazine; mannomustine; mitobromtol; mitolactol; pipobroman; gacytosine; arabinoside; 6-thioguanine; vinblastine; vincristine; vinorelbine; navelbine; novantrone; daunomycin; xeloda; ibandronate; CPT-11; difluoromethylornithine; retinoic acid; and the like.

In one embodiment, the drug candidate is a targeted therapy agent. As used herein, the term “targeted therapy agent” refers to any molecule which aims at one or more particular target molecules (such as, e.g., proteins) involved in tumor genesis, tumor progression, tumor metastasis, tumor cell proliferation, cell repair, and the like.

Suitable examples of targeted therapy agents include, but are not limited to, tyrosine-kinase inhibitors, serine/threonine kinase inhibitors, monoclonal antibodies and the like.

Suitable examples of targeted therapy agents include, but are not limited to, HER1/EGFR inhibitors (such as, e.g., brigatinib, erlotinib, gefitinib, olmotinib, osimertinib, rociletinib, vandetanib, and the like); HER2/neu inhibitors (such as, e.g., afatinib, lapatinib, neratinib, and the like); C-kit and PDGFR inhibitors (such as, e.g., axitinib, masitinib, pazopanib, sunitinib, sorafenib, toceranib, and the like): FLT3 inhibitors (such as, e.g., lestaurtinib, and the like); VEGFR inhibitors (such as, e.g., axitinib, cediranib, lenvatinib, nintedanib, pazopanib, regorafenib, semaxanib, sorafenib, sunitinib, tivozanib, toceranib, vandetanib, and the like); RET inhibitors (such as, e.g., vandetanib, entrectinib, and the like); c-MET inhibitors (such as, e.g., cabozantinib, and the like); bcr-abl inhibitors (such as, e.g., imatinib, dasatinib, nilotinib, ponatinib, radotinib, and the like); Src inhibitors (such as, e.g., bosutinib, dasatinib, and the like); Janus kinase inhibitors (such as, e.g., lestaurtinib, momelotinib, ruxolitinib, pacritinib, and the like); MAP2K inhibitors (such as, e.g., cobimetinib, selumetinib, trametinib, binimetinib, and the like); EML4-ALK inhibitors (such as, e.g., alectinib, brigatinib, ceritinib, crizotinib, and the like); Bruton's inhibitors (such as, e.g., ibrutinib, and the like); mTOR inhibitors (such as, e.g., everolimus, temsirolimus, and the like); hedgehog inhibitors (such as, e.g., sonidegib, vismodegib, and the like); CDK inhibitors (such as, e.g., palbociclib, ribociclib, and the like); anti-HER1/EGFR monoclonal antibodies (such as, e.g., cetuximab, necitumumab, panitumumab, and the like); anti-HER2/neu monoclonal antibodies (such as, e.g., ado-trastuzumab emtansine, pertuzumab, trastuzumab, trastuzumab-dkst, and the like); anti-EpCAM monoclonal antibodies (such as, e.g., catumaxomab, edrecolomab, and the like); anti-VEGF monoclonal antibodies (such as, e.g., bevacizumab, bevacizumab-awwb, and the like); anti-CD20 monoclonal antibodies (such as, e.g., ibritumomab, obinutuzumab, ocrelizumab, ofatumumab, rituximab, tositumomab, and the like); anti-CD30 monoclonal antibodies (such as, e.g., brentuximab, and the like); anti-CD33 monoclonal antibodies (such as, e.g., gemtuzumab, and the like); anti-CD52 monoclonal antibodies (such as, e.g., alemtuzumab, and the like); and antigen-binding fragments thereof.

In one embodiment, the drug candidate is a cytotoxic agent. As used herein, the term “cytotoxic agent” refers to any molecule that results in cell death by any mechanism.

Suitable examples of cytotoxic agents include, but are not limited to, taxanes, anthracyclines, alkylating agents, vinca alkaloids, antimetabolites, platinum agents, steroids, and chemotherapeutic agents. Suitable examples of taxanes include, but are not limited to, cabazitaxel, docetaxel, larotaxel, ortataxel, paclitaxel and tesetaxel. Suitable examples of anthracyclines include, but are not limited to, aclarubicin, amrubicin, daunorubicin, doxorubicin, epirubicin, idarubicin, pirarubicin, valrubicin and zorubicin. Suitable examples of alkylating agent include, but are not limited to, nitrogen mustards (such as, e.g., chlormethine, cyclophosphamide, ifosfamide, trofosfamide, chlorambucil, melphalan, prednimustine, bendamustine, uramustine, and the like), nitrosoureas (such as, e.g., carmustine, lomustine, semustine, fotemustine, nimustine, ranimustine, streptozocin, and the like), alkyl sulfonates (such as, e.g., busulfan, mannosulfan, treosulfan, and the like), aziridines (such as, e.g., carboquone, thiotepa, triaziquone, triethylenemelamine, benzodopa, meturedopa, uredopa, and the like), hydrazines (such as, e.g., procarbazine, and the like), triazenes (such as, e.g., dacarbazine, temozolomide, and the like), altretamine, mitobronitol, pipobroman, actinomycin, bleomycin, mitomycins and plicamycin. Suitable examples of vinca alkaloids include, but are not limited to, vinblastine, vincristine, vinflunine, vindesine and vinorelbine. Suitable examples of antimetabolites include, but are not limited to, antifolates (such as, aminopterin, methotrexate, pemetrexed, pralatrexate, raltitrexed, pemetrexed, and the like), purine analogues (such as, e.g., pentostatin, cladribine, clofarabine, fludarabine, nelarabine, tioguanine, mercaptopurine, and the like), pyrimidine analogues (such as, e.g., fluorouracil, capecitabine, doxifluridine, tegafur, tegafur/gimeracil/oteracil, carmofur, floxuridine, cytarabine, gemcitabine, azacytidine, decitabine, and the like), and hydroxycarbamide. Suitable examples of platinum agents include, but are not limited to, carboplatin, cisplatin, dicycloplatin, nedaplatin, oxaliplatin and satraplatin. Suitable examples of steroids include, but are not limited to, estrogen receptor modulators, androgen receptor modulators and progesterone receptor modulators. Suitable examples of chemotherapeutic agents have been described hereinabove.

In one embodiment, the drug candidate is a pro-apoptotic agent. As used herein, the term “pro-apoptotic agent” refers to any molecule able to induce apoptosis or programmed cell death in a cell upon administration.

Suitable examples of pro-apoptotic agents include, but are not limited to, histone deacetylase inhibitors (such as, e.g., sodium butyrate, depsipeptide and the like), bortezomib, deguelin, favopiridol, fenretinide, fludarabine, kaempferol, miltefosine, narciclasine, obatoclax, oblimersen, and oncrasin.

In one embodiment, the drug candidate is an anti-angiogenic agent. As used herein, the term “anti-angiogenic agent” refers to a molecule that reduces or prevents angiogenesis, which is responsible for the growth and development of blood vessels.

Suitable examples of anti-angiogenic agents include, but are not limited to, inhibitors of any of the vascular endothelial growth factor VEGF-A, VEGF-B, VEGF-C, or VEGP-D, which are major inducers of angiogenesis in normal and pathological conditions, and are essential in embryonic vasculogenesis. Additionally or alternatively. an anti-angiogenic agent also can inhibit other angiogenic factors, such as, without limitation, a member of the fibroblast growth factor (FGF) family such as FGF-1 (acidic), FGF-2 (basic), FGF-4 or FGF-5; or angiopoietin-1, a factor that signals through the endothelial cell-specific Tie2 receptor tyrosine kinase; or the receptor of any of these angiogenic factors.

In one embodiment, the gastrointestinal tract sample is incubated with drug candidates for several days, such as 1, 2, 3, 4, 5, 6, 7 days or more, preferably for three days.

In one embodiment, the method comprises then a step of counting the number of living versus dead cells and concluding that the drug candidate is efficient against the cancer cells from the gastrointestinal tract tumor biopsy sample if a majority of cells are dead.

By “majority”, it is meant at least 50% of the total cells in the sample, such as 50%, 55%, 60%, 65%, 70%, 75%, 80%, 85%, 90%, 95% or more of the total cells in the sample.

Additionally or alternatively, the method comprises a step of counting the number of living versus dead cells and concluding that the drug candidate is efficient against the cancer cells from the gastrointestinal tract tumor biopsy sample if the number of dead cells in the sample incubated with one or several drug candidates is statistically significantly higher than the number of dead cells in a control sample without drug candidate.

Additionally or alternatively, the method comprises a step of counting the number of living versus dead cells and concluding that the drug candidate is efficient against the cancer cells from the gastrointestinal tract tumor biopsy sample if the percentage of dead cells in the sample incubated with one or several drug candidates is statistically significantly higher than the percentage of dead cells in a control sample without drug candidate.

$$Percentage of dead cells = \frac{number of dead cells}{total number of cells}$$

By “statistically significantly”, it is meant with a p-value below 0.05, such as below 0.05, 0.01, 0.005 or 0.001, for example as can be determined by ANOVA (ANalysis Of VAriance).

Additionally or alternatively, the method comprises a step of counting the number of living versus dead cells and concluding that the drug candidate is efficient against the cancer cells from the gastrointestinal tract tumor biopsy sample if the cell death ratio is higher than a positivity threshold.

$$Cell death ratio = \frac{percentage of dead cells in the sample incubated with the drug candidate ( s )}{percentage of dead cells in a control sample without drug candidate ( s )}$$

The positivity threshold can be readily determined by the skilled artisan, based on clinical data obtained for a given drug candidate using the method of percentiles (as described, e.g., in Bounaix Morand du Puch et al., 2016. J Transl Med. 14:10). In one embodiment, a drug candidate is considered as non-efficient if the cell death ratio is beyond the 25^{th} percentile; a drug candidate is considered as efficient if the cell death ratio is above the 25^{th} percentile.

In one embodiment, the drug candidate is considered as efficient against the cancer cells from the gastrointestinal tract tumor biopsy sample if the cell death ratio is above about 1.5, 2, 3, 4, 5, 6, 7, 8, 9, 10, 15, 20, 25, 30, 35, 40, 45, 50 or more.

Cells may be counted using any means available and known to the skilled artisan. For example, cells may be first contacted with a dye specific for living cells and/or with a dye specific for dead cells. Cells may then be fixed and counted by eye or using a computed-implemented automated or semi-automated process, based on the color transmitted or emitted by the dye, or may alternatively be counted by flow cytometry.

In one embodiment, the dye is a fluorescent label. Examples of fluorescent labels specific for living cells include, without limitation, calcein acetoxymethyl ester (calcein AM; including calcein green AM, calcein blue AM, calcein violet AM, and calcein red-orange AM), fluorescein diacetate (FDA), carboxyfluorescein diacetate (CFDA), sulfofluorescein diacetate (SFDA), BCECF acetoxymethyl ester (BCECF AM), carboxynaphthofluorescein diacetate, chloromethyl SNARF-1 acetate, dihydrorhodamine 123, SYBR®14, SYTO™ 9, C12-resazurin, alamarBlue™.

Examples of fluorescent labels specific for dead cells include, without limitation, ethidium homodimer-1 (EthD-1), propidium iodide (PI), ethidium bromide, SYTOX® Green, SYTOX® Red, SYTOX® Orange, SYTOX® Blue, 7-aminoactinomycin D (7-AAD), POPO®-1, BOBO®-1, YOYO®-1, TOTO®-1, JOJO®-1, POPO®-3, LOLO®-1, BOBO®-3, YOYO®-3, TOTO®-3, PO-PRO®-1, YO-PRO®-1, TO-PRO®-1, JO-PRO®-1, PO-PRO®-3, YO-PRO®-3, TO-PRO®-3, TO-PRO®-5, Zombie Aqua™, Zombie Green™, Zombie NIR™, Zombie Red™, Zombie Violet™, Zombie UV™, Zombie Yellow™.

A preferred combination of living cell-specific fluorescent label and dead cell-specific fluorescent label is calcein AM/EthD-1.

However, when using two dyes, preferably two fluorescent labels (one specific for living cells and one specific for dead cells), the skilled artisan will readily know how to choose a suitable pair with different excitation and/or emission wavelengths so as to allow discrimination between living and dead cells.

In a fifth aspect, the invention relates to a kit-of-parts, suitable to implement the methods described herein.

In one embodiment, the kit-of-parts comprises:
optionally, a culture medium, such as a basal culture medium;
penicillin;
streptomycin;
gentamicin;
ciprofloxacin;
vancomycin;
metronidazole;
optionally, amphotericin B; and
optionally, a dye, preferably a fluorescent label, specific for living cells and/or a dye, preferably a fluorescent label, specific for dead cells, as described above.

In one embodiment, the kit-of-parts comprises:
a composition according to the present invention, comprising penicillin, streptomycin, gentamicin, ciprofloxacin, and vancomycin, optionally in a culture medium, such as in a basal culture medium;
metronidazole;
optionally, amphotericin B; and
optionally, a dye, preferably a fluorescent label, specific for living cells and/or a dye, preferably a fluorescent label, specific for dead cells, as described above.

In one embodiment, the kit-of-parts comprises:
a composition according to the present invention, comprising:
   optionally, a culture medium, such as a basal culture medium,
   penicillin, to a final concentration ranging from about 10 U/mL to about 1000 U/mL,
   streptomycin, to a final concentration ranging from about 10 μg/mL to about 1 mg/mL,
   gentamicin, to a final concentration ranging from about 10 μg/mL to about 1 mg/mL,
   ciprofloxacin, to a final concentration ranging from about 0.5 μg/mL to about 25 μg/mL, and
   vancomycin, to a final concentration ranging from about 5 μg/mL to about 125 μg/mL;
metronidazole;
optionally, amphotericin B; and
optionally, a dye, preferably a fluorescent label, specific for living cells and/or a dye, preferably a fluorescent label, specific for dead cells, as described above.

In one embodiment, the kit-of-parts comprises:
optionally, a composition according to the present invention, comprising:
   optionally, a culture medium, such as a basal culture medium,
   penicillin, to a final concentration of about 100 U/mL,
   streptomycin, to a final concentration of about 100 μg/mL,
   gentamicin, to a final concentration of about 100 μg/mL,
   ciprofloxacin, to a final concentration of about 2.5 μg/mL, and
   vancomycin, to a final concentration of about 25 μg/mL;
a composition according to the present invention, comprising:
   optionally, a culture medium, such as a basal culture medium,
   penicillin, to a final concentration of about 1000 U/mL,
   streptomycin, to a final concentration of about 1 mg/mL,
   gentamicin, to a final concentration of about 1 mg/mL,
   ciprofloxacin, to a final concentration of about 25 μg/mL, and
   vancomycin, to a final concentration of about 125 μg/mL;
a composition comprising a culture medium, such as a basal culture medium, and metronidazole to a final concentration ranging from about 1 μg/mL to about 200 μg/mL;
optionally, a composition comprising:
   optionally, a culture medium, such as a basal culture medium,
   penicillin, to a final concentration of about 500 U/mL;
   streptomycin, to a final concentration of about 500 μg/mL;
   gentamicin, to a final concentration of about 500 μg/mL;
   ciprofloxacin, to a final concentration of about 12.5 μg/mL; and
   vancomycin, to a final concentration of about 62.5 μg/mL;
optionally, a composition comprising:
   optionally, a culture medium, such as a basal culture medium,
   penicillin, to a final concentration of about 200 U/mL,
   streptomycin, to a final concentration of about 200 μg/mL,
   gentamicin, to a final concentration of about 200 μg/mL,
   ciprofloxacin, to a final concentration of about 5 μg/mL,
   vancomycin, to a final concentration of about 25 μg/mL, and
   amphotericin B, to a final concentration of about 2.5 μg/mL;
optionally, a dye, preferably a fluorescent label, specific for living cells and/or a dye, preferably a fluorescent label, specific for dead cells, as described above.

Also disclosed herein are:

E1: a composition comprising a basal medium, penicillin, streptomycin, gentamicin, ciprofloxacin, and vancomycin.

E2: the composition according to E1, which comprises:
a basal medium;
penicillin, to a final concentration ranging from about 10 U/mL to about 1000 U/mL;
streptomycin, to a final concentration ranging from about 10 μg/mL to about 1 mg/mL;
gentamicin, to a final concentration ranging from about 10 μg/mL to about 1 mg/mL;
ciprofloxacin, to a final concentration ranging from about 0.5 μg/mL to about 25 μg/mL; and
vancomycin, to a final concentration ranging from about 5 μg/mL to about 125 μg/mL.

E3: the composition according to E1 or E2, which comprises:
a basal medium;
penicillin, to a final concentration of about 100 U/mL;
streptomycin, to a final concentration of about 100 μg/mL;
gentamicin, to a final concentration of about 100 μg/mL;
ciprofloxacin, to a final concentration of about 2.5 μg/mL; and
vancomycin, to a final concentration of about 25 μg/mL.

E4: the composition according to E1 or E2, which comprises:
a basal medium;
penicillin, to a final concentration of about 1000 U/mL;
streptomycin, to a final concentration of about 1 mg/mL;
gentamicin, to a final concentration of about 1 mg/mL;
ciprofloxacin, to a final concentration of about 25 μg/mL; and
vancomycin, to a final concentration of about 125 μg/mL.

E5: the composition according to E1 or E2, which comprises:
a basal medium;
penicillin, to a final concentration of about 500 U/mL;
streptomycin, to a final concentration of about 500 μg/mL;
gentamicin, to a final concentration of about 500 μg/mL;
ciprofloxacin, to a final concentration of about 12.5 μg/mL; and
vancomycin, to a final concentration of about 62.5 μg/mL.

E6: the composition according to E1 or E2, which comprises:
a basal medium;
penicillin, to a final concentration ranging from about 50 U/mL to about 300 U/mL;
streptomycin, to a final concentration ranging from about 50 μg/mL to about 300 μg/mL;
gentamicin, to a final concentration ranging from about 50 μg/mL to about 300 μg/mL;
ciprofloxacin, to a final concentration ranging from about 1 μg/mL to about 7.5 μg/mL;
vancomycin, to a final concentration ranging from about 10 μg/mL to about 40 μg/mL; and
amphotericin B, to a final concentration ranging from about 1 μg/mL to about 4 μg/mL.

E7: the composition according to E6, which comprises:
a basal medium;
penicillin, to a final concentration of about 200 U/mL;
streptomycin, to a final concentration of about 200 μg/mL;
gentamicin, to a final concentration of about 200 μg/mL;
ciprofloxacin, to a final concentration of about 5 μg/mL;
vancomycin, to a final concentration of about 25 μg/mL; and
amphotericin B, to a final concentration of about 2.5 μg/mL.

E8: a method of decontaminating a gastrointestinal tract sample, comprising the steps of:
optionally, preserving the gastrointestinal tract sample in a preservation solution comprising the composition according to E1 or E2, preferably the composition according to E2, preferably for a period of time of at most about 55 hours and at a temperature ranging from about 2° C. to about 8° C.;
contacting the gastrointestinal tract sample with a first decontamination solution comprising a basal medium and metronidazole, preferably to a final concentration ranging from about 1 μg/mL to about 200 μg/mL, preferably for a period of time ranging from about 10 minutes to about 2 hours at a temperature ranging from about 20° C. to about 42° C. under a controlled atmosphere with a carbon dioxide (CO₂) concentration ranging from about 0.1% to about 20%; and
contacting the gastrointestinal tract sample with a second decontamination solution comprising the composition according to E1 or E2, preferably the composition according to E2, preferably for a period of time ranging from about 10 minutes to about 2 hours at a temperature ranging from about 20° C. to about 42° C. under a controlled atmosphere with a CO₂ concentration ranging from about 0.1% to about 20%, preferably this last step is repeated twice.

E9: the method of decontaminating a gastrointestinal tract sample according to E8, comprising the steps of:
optionally, preserving the gastrointestinal tract sample in a preservation solution comprising the composition according to E3, for a period of time ranging from a few minutes to about 55 hours and at a temperature ranging from about 2° C. to about 8° C.;
contacting the gastrointestinal tract sample with a first decontamination solution comprising a basal medium and metronidazole to a final concentration of about 20 μg/mL, for a period of time ranging from about 10 minutes to about 2 hours at a temperature ranging from about 35° C. to about 42° C. under a controlled atmosphere with a carbon dioxide (CO₂) concentration ranging from about 1% to about 10%; and
contacting the gastrointestinal tract sample with a second decontamination solution comprising the composition according to E4, for a period of time ranging from about 10 minutes to about 2 hours at a temperature ranging from about 35° C. to about 42° C. under a controlled atmosphere with a CO₂ concentration ranging from about 1% to about 10%, wherein this last step is repeated twice.

E10: a method of culturing a gastrointestinal tract sample, comprising the steps of:
performing the method of decontaminating a gastrointestinal tract sample according to E8 or E9;
optionally, mechanically and/or enzymatically dissociating the gastrointestinal tract sample; and
culturing the gastrointestinal tract sample in a culture medium comprising the composition according to E6, preferably for several days at a temperature ranging from about 20° C. to about 42° C. under a controlled atmosphere with a CO₂ concentration ranging from about 0.1% to about 20%.

E11: the method of culturing a gastrointestinal tract sample according to E10, comprising the steps of:
performing the method of decontaminating a gastrointestinal tract sample according to E8 or E9;
mechanically and enzymatically dissociating the gastrointestinal tract sample, wherein enzymatically dissociating the gastrointestinal tract sample is carried out in the composition according to E5 supplemented with at least one enzyme capable of releasing cells from a tissue, preferably supplemented with type II collagenase and trypsin; and
culturing the gastrointestinal tract sample in a culture medium comprising the composition according to E7, for several days at a temperature ranging from about 35° C. to about 42° C. under a controlled atmosphere with a CO₂ concentration ranging from about 1% to about 10%.

E12: a method of evaluating the efficacy of one or several drug candidates against a gastrointestinal tract cancer or tumor, comprising the steps of:
performing the method of culturing a gastrointestinal tract sample according to E10 or E11, wherein the gastrointestinal tract sample is a gastrointestinal tract cancer or tumor sample;
contacting the gastrointestinal tract sample with the one or several drug candidates;
counting the number of dead cells and optionally, determining a percentage or ratio of dead cells in the gastrointestinal tract sample; and
concluding that the one or several drug candidates is efficient against the gastrointestinal tract cancer or tumor based on the number, percentage and/or ratio of dead cells in the gastrointestinal tract sample.

E13: the method of evaluating the efficacy of one or several drug candidates against a gastrointestinal tract cancer or tumor according to E12, wherein the step of counting the number of dead cells first comprises contacting the gastrointestinal tract sample with a dye, preferably a fluorescent label, specific for living cells, and/or a dye, preferably a fluorescent label, specific for dead cells, and then counting the number of living and/or dead cells based on the color transmitted or emitted by the dye, preferably by the fluorescent label.

E14: the methods according to any one of E8 to E13, wherein the gastrointestinal tract sample is a colorectal cancer or tumor sample.

E15: a kit-of-parts comprising:
optionally, a preservation medium comprising the composition according to E3;
metronidazole,
a decontamination medium comprising the composition according to E4;
optionally, a dissociation medium comprising the composition according to E5;
optionally, at least one enzyme capable of releasing cells from a tissue;
optionally, a culture medium comprising the composition according to E7; and
optionally, a dye, preferably a fluorescent label, specific for living cells and/or a dye, preferably a fluorescent label, specific for dead cells.

### EXAMPLES

The present invention is further illustrated by the following examples.

### Example 1

### Materials and Methods

### Colorectal Tumor Sample

Colorectal tumor samples of >1 cm³ were obtained shortly after surgical resection (time between resection and culture <55 hours). Colorectal tumor samples were not fixed, not frozen, and maintained between 2° C. and 8° C. in a medium containing an OncoMiD-Via solution.

OncoMiD-Via comprises a culture medium comprising a DMEM/F-12 basal medium supplemented with 0.1% of a 30% (300 mg/mL) BSA stock solution and antimicrobial agents, as detailed below.

### Step 1 (D0)

Decontamination of the tumor sample was carried out in solutions comprising a culture medium comprising a DMEM/F-12 basal medium and antimicrobial agents, as detailed below.

After decontamination, the colorectal tumor sample was cut into fragments of 5-10 mm³ using a tissue chopper.

The chopped tumor sample was then contacted with a dissociation solution comprising type II collagenase and trypsin (as described in EP 1 795 897 B1), supplemented with antimicrobial agents, as detailed below, for tumor cell dissociation in a rotator for 1h30 to 2h30.

Finally, the tumor cells were placed in culture in a culture medium comprising an OncoMiD solution of culture medium comprising a DMEM/F-12 basal medium, similar to that described in EP 1 795 897 B1, supplemented with antimicrobial agents, as detailed below.

### Step 2 (D5±1)

The culture medium was refreshed after 5 days±1.

### Step 3 (D7±1)

Cultured tumor cells were counted at D7±1, and contacted with drug candidates or combinations thereof.

Step 4 (Step 3±3 days)

Three days after step 3, live versus dead tumor cells were detected by fluorescence using Calcein AM (green fluorescence of living cells) and ethidium homodimer-1 (red fluorescence of dead cells).

### Results

Preliminary tests and searches on antimicrobial agents led to the identification of 7 antimicrobial agents which could possibly be useful to decontaminate colorectal tumor samples: penicillin, streptomycin, gentamicin, ciprofloxacin, vancomycin, metronidazole, and amphotericin B.

A first test was carried out on a colorectal tumor sample, maintained in a medium containing an OncoMiD-Via solution with the following antimicrobial agents:
penicillin, final concentration: 100 U/mL
streptomycin, final concentration: 100 μg/mL
gentamicin, final concentration: 100 μg/mL
ciprofloxacin, final concentration: 2.5 μg/mL
vancomycin, final concentration: 25 μg/mL
metronidazole, final concentration: 10 μg/mL
amphotericin B, final concentration: 1.25 μg/mL

Since the sample showed bacterial contamination in spite of this mix of 7 antimicrobial agents, we decided to sequentially contact the colorectal tumor sample with three different decontaminating solutions, upon reception of the sample and prior to tumor cell dissociation and culture. The decontaminating solutions contained the same mix of antimicrobial agents, but at higher concentrations:
decontaminating solution #1 (twice 5 minutes at 37° C. under 5% CO₂) contained DMEM/F-12 with penicillin (final concentration 1000 U/mL), streptomycin (final concentration 1 mg/mL), gentamicin (final concentration 1 mg/mL), ciprofloxacin (final concentration 25 μg/mL), vancomycin (final concentration 125 μg/mL), metronidazole (final concentration 100 μg/mL), and amphotericin B (final concentration 12.5 μg/mL);
decontaminating solution #2 (once 5 minutes at 37° C. under 5% CO₂) contained DMEM/F-12 with penicillin (final concentration 400 U/mL), streptomycin (final concentration 400 μg/mL), gentamicin (final concentration 400 μg/mL), ciprofloxacin (final concentration 10 μg/mL), vancomycin (final concentration 50 μg/mL), metronidazole (final concentration 40 μg/mL), and amphotericin B (final concentration 5 μg/mL);
decontaminating solution #3 (once 5 minutes at 37° C. under 5% CO₂) contained DMEM/F-12 with penicillin (final concentration 200 U/mL), streptomycin (final concentration 200 μg/mL), gentamicin (final concentration 200 μg/mL), ciprofloxacin (final concentration 5 μg/mL), vancomycin (final concentration 25 μg/mL), metronidazole (final concentration 20 μg/mL), and amphotericin B (final concentration 2.5 μg/mL).

Following these 4 decontamination baths, the tumor cell sample was dissociated and cultured. However, bacterial contamination was still observed.

We suspected a possible antagonism between some or all of the antimicrobial agents when mixed together, a phenomenon that had already been observed back in the 1960's (Manten & Wisse, 1961. Nature. 192:671-2). We also hypothesized that a total time of 20 minutes may not be sufficient to allow full effect of the antimicrobial agents.

A second test was thus carried out on a colorectal tumor sample, maintained in a medium containing an OncoMiD-Via solution as described above, except that the solution did not comprise metronidazole and amphotericin B. Upon reception of the sample, it was sequentially contacted with two different decontamination solutions as follows:
decontaminating solution #1 (once 30 minutes at 37° C. under 5% CO₂) contained DMEM/F-12 with metronidazole (final concentration 20 μg/mL);
decontaminating solution #2 (twice 30 minutes at 37° C. under 5% CO₂) contained DMEM/F-12 with penicillin (final concentration 1000 U/mL), streptomycin (final concentration 1 mg/mL), gentamicin (final concentration 1 mg/mL), ciprofloxacin (final concentration 25 μg/mL), and vancomycin (final concentration 125 μg/mL).

After these three decontamination baths, the colorectal tumor sample was cut into fragments, contacted with a dissociation solution (as described in EP 1 795 897 B1), supplemented with penicillin (final concentration 500 U/mL), streptomycin (final concentration 500 μg/mL), gentamicin (final concentration 500 μg/mL), ciprofloxacin (final concentration 12.5 μg/mL), and vancomycin (final concentration 62.5 μg/mL). The dissociated colorectal tumor sample-a tumor cell sample at this stage-was then cultured in a culture medium comprising one of the two following OncoMiD solutions of culture medium comprising a DMEM/F-12 basal medium and antimicrobial agents:
“OncoMiD 1×”: DMEM/F-12 with penicillin (final concentration 200 U/mL), streptomycin (final concentration 200 μg/mL), gentamicin (final concentration 200 μg/mL), ciprofloxacin (final concentration 5 μg/mL), vancomycin (final concentration 25 μg/mL), and amphotericin B (final concentration 2.5 μg/mL); or
“OncoMiD 2×”: DMEM/F-12 with penicillin (final concentration 400 U/mL), streptomycin (final concentration 400 μg/mL), gentamicin (final concentration 400 μg/mL), ciprofloxacin (final concentration 10 μg/mL), vancomycin (final concentration 50 μg/mL), and amphotericin B (final concentration 5 μg/mL).

No contamination was observed in the cultured colorectal tumor sample, using either “OncoMid 1×” or “OncoMid 2×”. Surprisingly though, tumor cells cultured in OncoMid 2× (i.e., comprising twice as much antimicrobial agents as OncoMid 1×) showed a lower viability than those cultured in OncoMid 1×.

### Conclusion

To summarize, we have thus developed an efficient method of decontaminating colorectal tumor samples, which are known to be prone to important microbial infections.

The method first comprises storing the tumor cell sample immediately after resection from a patient in a solution comprising a culture medium comprising a DMEM/F-12 basal medium supplemented with antimicrobial agents (penicillin, streptomycin, gentamicin, ciprofloxacin, and vancomycin), preferably for at most 55 hours between the time of resection and the culture of the cells, without fixation, without freezing, and at a temperature between about 2° C. and about 8° C.

Upon reception of the sample, it is immersed in three decontamination baths, for about 30 minutes/bath, at around 37° C. under 5% CO₂ (one comprising metronidazole, and two comprising penicillin, streptomycin, gentamicin, ciprofloxacin, and vancomycin).

The sample is then cut into fragments, and contacted with a dissociation solution (as described in EP 1 795 897 B1) supplemented with antimicrobial agents (penicillin, streptomycin, gentamicin, ciprofloxacin, and vancomycin). Once dissociation is fully achieved (by visual assessment), the tumor cells are placed in culture in a culture medium comprising a DMEM/F-12 basal medium, similar to that described in EP 1 795 897 B1, supplemented with antimicrobial agents (penicillin, streptomycin, gentamicin, ciprofloxacin, vancomycin and amphotericin B).

The decontamination and culture protocol described herein can be readily applied to any gastrointestinal tract sample, in particular to any colorectal sample, and not just to cancer or tumor samples. However, when the sample is a cancer or tumor sample, this protocol can be particularly useful for the preparation of a chemosensitivity assay.

1-19. (canceled)

20. A composition comprising penicillin, streptomycin, gentamicin, ciprofloxacin, and vancomycin.

21. The composition according to claim 20, further comprising a culture medium.

22. The composition according to claim 21, wherein the culture medium is devoid of animal-derived serum or proteins thereof.

23. The composition according to claim 21, wherein the culture medium is supplemented with animal-derived serum or proteins thereof.

24. The composition according to claim 20, comprising:
penicillin, to a final concentration ranging from about 10 U/mL to about 1000 U/mL;
streptomycin, to a final concentration ranging from about 10 μg/mL to about 1 mg/mL;
gentamicin, to a final concentration ranging from about 10 μg/mL to about 1 mg/mL;
ciprofloxacin, to a final concentration ranging from about 0.5 μg/mL to about 25 μg/mL; and
vancomycin, to a final concentration ranging from about 5 μg/mL to about 125 μg/mL.

25. The composition according to claim 20, comprising:
penicillin, to a final concentration of about 100 U/mL;
streptomycin, to a final concentration of about 100 μg/mL;
gentamicin, to a final concentration of about 100 μg/mL;
ciprofloxacin, to a final concentration of about 2.5 μg/mL; and
vancomycin, to a final concentration of about 25 μg/mL.

26. The composition according to claim 20, comprising:
penicillin, to a final concentration of about 1000 U/mL;
streptomycin, to a final concentration of about 1 mg/mL;
gentamicin, to a final concentration of about 1 mg/mL;
ciprofloxacin, to a final concentration of about 25 μg/mL; and
vancomycin, to a final concentration of about 125 μg/mL.

27. The composition according to claim 20, comprising:
penicillin, to a final concentration of about 500 U/mL;
streptomycin, to a final concentration of about 500 μg/mL;
gentamicin, to a final concentration of about 500 μg/mL;
ciprofloxacin, to a final concentration of about 12.5 μg/mL; and
vancomycin, to a final concentration of about 62.5 μg/mL.

28. The composition according to claim 27, further comprising at least one tissue-dissociation enzyme.

29. The composition according to claim 20, comprising:
penicillin, to a final concentration ranging from about 50 U/mL to about 300 U/mL;
streptomycin, to a final concentration ranging from about 50 μg/mL to about 300 μg/mL;
gentamicin, to a final concentration ranging from about 50 μg/mL to about 300 μg/mL;
ciprofloxacin, to a final concentration ranging from about 1 μg/mL to about 7.5 μg/mL;
vancomycin, to a final concentration ranging from about 10 μg/mL to about 40 μg/mL; and
amphotericin B, to a final concentration ranging from about 1 μg/mL to about 4 μg/mL.

30. The composition according to claim 29, comprising:
penicillin, to a final concentration of about 200 U/mL;
streptomycin, to a final concentration of about 200 μg/mL;
gentamicin, to a final concentration of about 200 μg/mL;
ciprofloxacin, to a final concentration of about 5 μg/mL;
vancomycin, to a final concentration of about 25 μg/mL; and
amphotericin B, to a final concentration of about 2.5 μg/mL.

31. A method of decontaminating a gastrointestinal tract sample, comprising the steps of:
contacting the gastrointestinal tract sample with a first decontamination solution comprising metronidazole; and
contacting the gastrointestinal tract sample with a second decontamination solution comprising the composition according to claim 20.

32. The method according to claim 31, wherein the contacting step of step (a) is carried out for a period of time ranging from about 10 minutes to about 2 hours at a temperature ranging from about 20° C. to about 42° C. under a controlled atmosphere with a carbon dioxide (CO₂) concentration ranging from about 0.1% to about 20%.

33. The method according to claim 31, wherein the contacting step of step (b) is carried out for a period of time ranging from about 10 minutes to about 2 hours at a temperature ranging from about 20° C. to about 42° C. under a controlled atmosphere with a carbon dioxide (CO₂) concentration ranging from about 0.1% to about 20%.

34. The method according to claim 31, comprising the steps of:
contacting the gastrointestinal tract sample with a first decontamination solution comprising metronidazole to a final concentration of about 20 μg/mL, for a period of time ranging from about 10 minutes to about 2 hours at a temperature ranging from about 35° C. to about 42° C. under a controlled atmosphere with a carbon dioxide (CO₂) concentration ranging from about 1% to about 10%; and
contacting the gastrointestinal tract sample with a second decontamination solution comprising the composition, for a period of time ranging from about 10 minutes to about 2 hours at a temperature ranging from about 35° C. to about 42° C. under a controlled atmosphere with a carbon dioxide (CO₂) concentration ranging from about 1% to about 10%, wherein step (b) is repeated twice,
wherein the composition comprises:
the penicillin, to a final concentration of about 1000 U/mL,
streptomycin, to a final concentration of about 1 mg/mL,
gentamicin, to a final concentration of about 1 mg/mL,
ciprofloxacin, to a final concentration of about 25 μg/mL, and
vancomycin, to a final concentration of about 125 μg/mL.

35. The method according to claim 31, further comprising the step of culturing said gastrointestinal tract sample in a culture medium comprising a composition comprising:
penicillin, to a final concentration ranging from about 50 U/mL to about 300 U/mL;
streptomycin, to a final concentration ranging from about 50 μg/mL to about 300 μg/mL;
gentamicin, to a final concentration ranging from about 50 μg/mL to about 300 μg/mL;
ciprofloxacin, to a final concentration ranging from about 1 μg/mL to about 7.5 μg/mL;
vancomycin, to a final concentration ranging from about 10 μg/mL to about 40 μg/mL; and
amphotericin B, to a final concentration ranging from about 1 μg/mL to about 4 μg/mL.

36. The method according to claim 35, further comprising the steps of:
mechanically and enzymatically dissociating the gastrointestinal tract sample, wherein enzymatically dissociating the gastrointestinal tract sample is carried out in a composition comprising:
penicillin, to a final concentration of about 500 U/mL,
streptomycin, to a final concentration of about 500 μg/mL,
gentamicin, to a final concentration of about 500 μg/mL,
ciprofloxacin, to a final concentration of about 12.5 μg/mL,
vancomycin, to a final concentration of about 62.5 μg/mL, and
at least one tissue-dissociation enzyme; and
culturing the gastrointestinal tract sample in a culture medium comprising a composition comprising:
penicillin, to a final concentration of about 200 U/mL,
streptomycin, to a final concentration of about 200 μg/mL,
gentamicin, to a final concentration of about 200 μg/mL,
ciprofloxacin, to a final concentration of about 5 μg/mL,
vancomycin, to a final concentration of about 25 μg/mL, and
amphotericin B, to a final concentration of about 2.5 μg/mL,
for several days at a temperature ranging from about 35° C. to about 42° C. under a controlled atmosphere with a carbon dioxide (CO₂) concentration ranging from about 1% to about 10%.

37. A method of evaluating the efficacy of one or several drug candidates against a gastrointestinal tract cancer or tumor, comprising the steps of:
performing the method of culturing a gastrointestinal tract sample according to claim 35, wherein the gastrointestinal tract sample is a gastrointestinal tract cancer or tumor sample;
contacting the gastrointestinal tract sample with the one or several drug candidates;
counting the number of dead cells; and
concluding that the one or several drug candidates is efficient against the gastrointestinal tract cancer or tumor based on the number, percentage and/or ratio of dead cells in the gastrointestinal tract sample.

38. The method according to claim 37, wherein step (c) first comprises contacting the gastrointestinal tract sample with a dye specific for living cells, and/or a dye specific for dead cells, and then counting the number of living and/or dead cells based on the color transmitted or emitted by the dye.

39. A kit-of-parts comprising:
metronidazole;
a decontamination medium comprising a composition according to claim 24.
